(19) United States
(12) Patent Application Publication
(10) Pub. No.: US 2004/0115667 A1
(43) Pub. Date: Jun. 17, 2004

(54) Dna-sequences, which code for an apoptosis signal transduction protein

(76) Inventors: Jurg Tschopp, Epalinges (CH); Kay Hofmann, Kohln (DE)

Correspondence Address: CONNOLLY BOVE LODGE & HUTZ, LLP, P O BOX 2207, WILMINGTON, DE, 19899, US

(21) Appl. No.: 10/450,366

(22) PCT Filed: Dec. 12, 2001
(86) PCT No.: PCT/EP01/14597

(30) Foreign Application Priority Data
Dec. 12, 2000 (DE) 10061766.2

(51) Int. Cl. C12Q 1/68; C07H 21/04

(52) U.S. Cl. 435/6; 435/69.1; 435/320.1; 435/325; 530/350; 536/23.2

(57) ABSTRACT

The invention relates to DNA sequences, which code for an apoptotic signal transduction protein (Bcl-Rambo) or for polypeptides with a domain of a protein of this type (BHNo domains of Bcl-Rambo), to expression vectors, host cells, genetic products of the aforementioned sequences and antibodies against said genetic products. The invention also relates to an expression and isolation method, to compounds that modulate the apoptotic action, to a method for identifying compounds of this type and to the use of said compounds, or the DNA sequences, or genetic products as medicaments.

The present invention relates to DNA sequences which encode an apoptotic signal transduction protein (Bcl-rambo) or polypeptides containing a domain of such a protein (BHNo domain of Bcl-rambo), to expression vectors, to host cells, to gene products of the abovementioned sequences, to antibodies directed against these gene products, to methods for expression and isolation, to compounds which modulate the apoptotic effect, to methods for identifying such compounds, and to the use of these compounds or the DNA sequences or gene products as pharmaceuticals.

In addition to necrosis, which represents one form of cell death, the prior art also discloses apoptosis as being another form of cell death. Apoptosis is a physiological process which intervenes in a regulatory manner in many physiological processes in multicellular organisms. Thus, for example, apoptosis occurs in embryonic development, plays a role in tissue or organ morphology and intervenes in the modulation of the immune response and is consequently, speaking in general terms, of central importance for homeostasis in multicellular organisms (Wyllie et al. (1980) Rev. Cytol. 68 (251), 251-251). In recent years, various apoptotic signal transduction pathways have been elucidated in the prior art, for example FasL-induced cell apoptosis, which, after having been triggered extracellularly, is transmitted intracellularly by a cascade of capases which have to be activated. The studies of Adams et al. ((1998) Science 281, 1322-1326) and Gross et al. ((1999) Genes Dev 13 (15), 1899-911) show not only, as has been known for a long time from the prior art, that proteins of the Bcl-2 family are involved in regulating apoptosis but also that these proteins belong to three different subfamilies. While Bcl-2 and other close relatives of Bcl-2, including Bcl-x, Bcl-w and C. Elegans CED-9, inhibit apoptosis and establish their own subfamily, there are two other subfamilies which, as has been shown, have precisely the opposite effect, i.e. promote cell death. These subfamilies are, on the one hand, the subfamily containing proteins such as Bax, Bak and Bok and, on the other hand, the third subfamily to which the following proteins belong: Bad, Bik/Nbk, Blk, Hrk, Bid, Bim/Bod and Noxa or the C. Elegans protein EGL/1. The two subfamilies to which, on the one hand, Bcl-2 belongs, as an inhibitor of apoptosis, and, on the other hand, Bax belongs, as an activator of apoptosis, are jointly characterized by three out of four conserved “BH” (abbreviation for Bcl-2 homology) sequence motifs. In contrast to this in the case of the third subfamily, which is represented, for example, by the mammalian proteins Bad, Bik/Nbk, Blk, Hrk, Bid, Bim/Bod and Noxa or the C. Elegans EGL/1 protein, there is only one homology, in the form of the short BH3 sequence motif, with the two previously mentioned subfamilies, for which reason the state of the art designates proteins of this third subfamily as being “BH3-only” proteins. It is furthermore known that the proteins which belong to the two subfamilies having an antiapoptotic effect are characterized by a hydrophobic sequence segment which is located C-terminally and which enables the proteins to interact with the endoplasmic reticulum, the nuclear envelope or the outer mitochondrial membrane. Thus, these proteins, which promote the survival of the cell, are typically membrane-bound in contrast to other proteins which are involved in apoptosis.

Finally, it is known, from the studies of Oltvai et al. ((1993) Cell 74(4), 609-619; (1994) Cell 79 (2), 189-92) that the relative concentrations, which are prevailing in the cell at the time, of the proteins which belong to subfamilies having opposing effects determine whether the cell should die or survive. The fact that the proteins which have opposing effects, and which are derived from the respective subfamilies, i.e. proteins having pro-apoptotic effect or having an antiapoptotic effect, can form heterodimers which are able to neutralize their respective opposing effects, could play a role in this connection. In this regard, it has still not been clarified whether, for example, in order to display its apoptosis-inhibitory effect, Bcl-2 has necessarily to bind pro-apoptotic family members, for example Bax or Bak, or whether the binding to these pro-apoptotic proteins is not crucial for the effect of Bcl-2 (Yin et al. (1994) Nature 369 (6478), 321-323; Cheng et al. (1996) Nature 379 (6565), 554-556).

For the reasons expanded, apoptotic signal transduction, the interaction between Bcl-2 homologs belonging to different subfamilies, and the entirety of the proteins involved in regulating this apoptotic signal transduction pathway, are therefore still completely inadequately elucidated in the prior art. For this reason, it is not possible either, in accordance with the prior art, to use therapeutic measures to selectively modulate the apoptotic events in the cell.

The object of the present invention is, on the one hand, to elucidate apoptotic signal transduction mechanisms and to identify those proteins (together with their amino acid sequences), and the underlying DNA sequences, which are involved in apoptotic signal transduction and, on the other hand, based on these insights, and with the aid, where appropriate, of pathophysiological findings, to be able to make available substances, or their use, for treating diseases or disturbances which relate to apoptotic signal transduction.

These objects are achieved, within the context of the present invention, by substances, methods and uses as described in claims 1, 5, 6, 8, 11, 14, 15, 16, 19, 23, 25, 26, 27, 28 and 29. Particular advantageous embodiments are described in the pertinent subclaims.

In order to achieve these objects, the inventors first of all established that proteins possessing what is termed a BHNo domain play a crucial role in the intracellular transmission or induction of an apoptotic signal. In addition, these proteins can, where appropriate, exhibit BH sequence motifs, for example at least one BH1, BH2, BH3 and/or BH4 sequence motif. This thereby makes available, according to the invention, proteins which are involved in the apoptotic signal cascade and which belong to a further subfamily of apoptotic signal transduction proteins which is unknown in the state of the art.

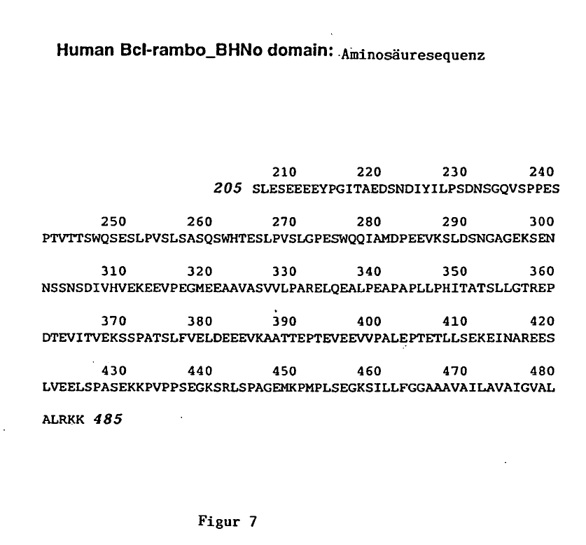

The present invention relates, therefore, to DNA sequences which contain a sequence region which encodes a polypeptide having an amino acid sequence as depicted in FIG. 7 (BHNo domain), including all the derivatives, fragments or alleles which are functionally homologous. In particular, the invention also includes all the DNA sequences which hybridize with the DNA sequences according to the invention, including the sequences which are in each case complementary in the double strand (claim 1).

In another preferred embodiment, the invention discloses DNA sequences whose gene product encodes a polypeptide as depicted in FIG. 7, including all the functionally homologous derivatives, alleles or fragments of such a DNA sequence and all the nonfunctional derivatives, alleles, analogs or fragments which are able to inhibit the apoptotic signal cascade. The invention also discloses DNA sequences (including the sequences of the complementary DNA strand) which hybridize with these DNA sequences according to the invention (claim 2). These derivatives, analogs, fragments or alleles are prepared using standard methods (Sambrook et al. 1989, Molecular Cloning: A Laboratory Manual, Cold Spring Harbor, N.Y.). In this connection, one or more codons is/are inserted, omitted or replaced in the DNA sequences which encode a protein which contains a BHNo domain in order to obtain a polypeptide which exhibits a difference, in relation to at least one amino acid, as compared with the native sequence.

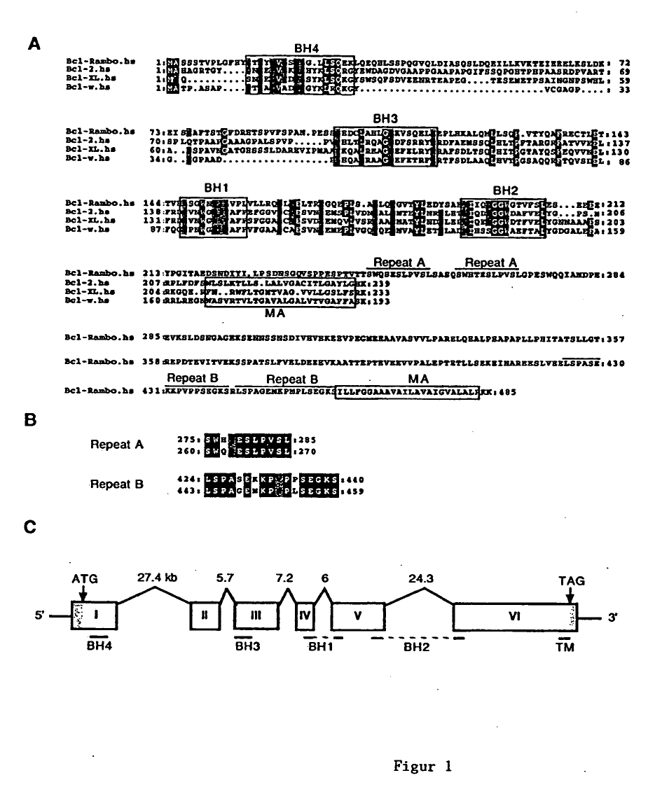

The inventive subject matter of the present invention also includes constituent DNA sequences which encode segments of the BHNo domain, in particular encode the constituent sequence from AA 213 to AA 459 or up to an AA which is located further N-terminally, in particular, in addition, encode the constituent sequence from AA 245 to AA 459 or up to an AA which is located further N-terminally, in addition, in particular, also encode the constituent sequence from AA 285 to AA 459 or up to an AA which is located further N-terminally, and, in particular, in addition, encode a constituent sequence from AA 431 to AA 459 or up to an AA which is located further N-terminally. With regard to the respective AA sequences, the reader is. referred to the numbering shown in FIG. 1A. Speaking in general terms, the invention discloses DNA sequences which contain at least one DNA sequence which encode(s) a constituent segment, which is at least 8, preferably at least 12, even more preferably at least 20, AA in length, of the BHNo domain (AA 205 to AA 485 in accordance with FIG. 1A), including all the derivatives, analogs or alleles. The invention also discloses the AA sequences which ensue from these DNA sequences according to the invention. Very particular preference is given to the DNA sequences (repeat sequences) which underlie the AA sequences shown in FIG. 1B or to DNA sequences which contain segments which encode these AA sequences, including all the derivatives, alleles and analogs. The invention also discloses the oligopeptide or polypeptide sequences which can be obtained using the abovementioned DNA sequences.

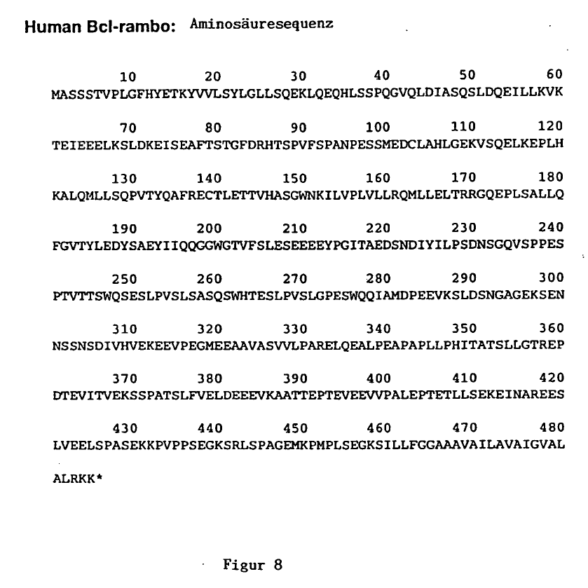

Preference is furthermore given to DNA sequences which contain a sequence region which encode a polypeptide as depicted in FIG. 8, including all the functionally homologous derivatives, alleles or fragments, or including all the sequences which hybridize with the sequences (claim 3). The hybridization conditions are preferably selected such that hybridization takes place at 40° C., more preferably at 50° C. and even more preferably at 60° C.

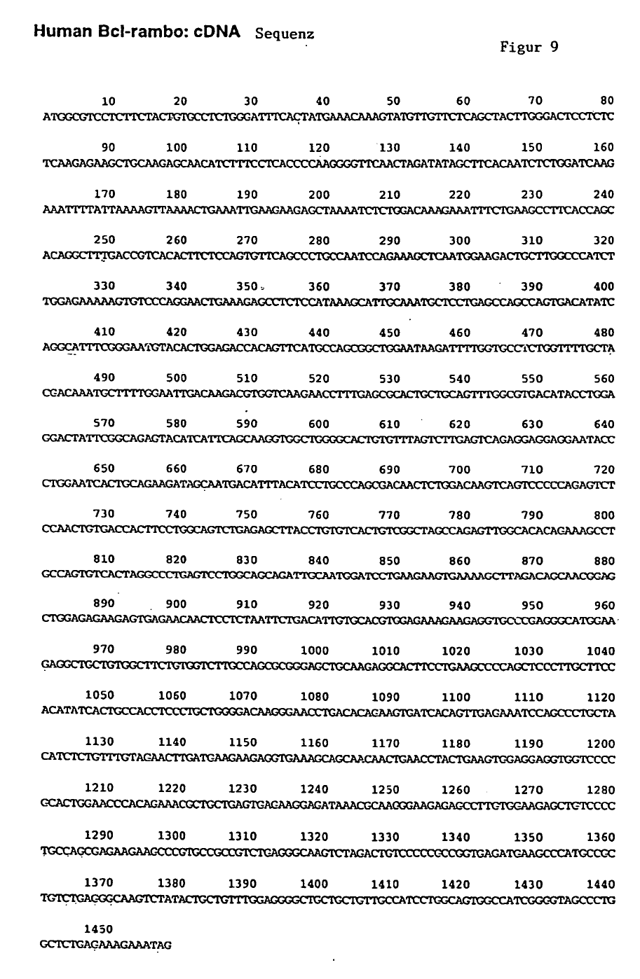

Preference is furthermore given to DNA sequences which contain one of the (c)DNA sequences specified in FIG. 9 (claim 4).

In addition, the invention also discloses all the DNA sequences which encode a protein which essentially corresponds to the Bcl-rambo protein. These DNA sequences contain only a small number of changes as compared with the sequence specified in FIG. 9, for example the sequences can be isoforms. The number of the sequence changes will typically be not greater than 10. Such DNA sequences, which essentially correspond to the DNA sequence encoding Bcl-rambo and which also encode a biologically active protein, can be obtained by means of well-known mutagenesis methods, and the biological activity of the proteins encoded by the mutants can be identified by means of screening methods, for example binding studies or the ability to induce apoptosis. The relevant mutagenesis methods include site-directed mutagenesis, which provides for the automatically implemented synthesis of a primer containing at least one base change. After the polymerization reaction, the heteroduplex vector is transferred into a suitable cell system (e.g. E. coli) and correspondingly transformed clones are isolated. All the methods, with which the skilled person is familiar and which can be carried out in vivo, in situ, or in vitro, for preparing, modifying and/or detecting DNA sequences according to the invention also come into consideration (PCR (Innis et al. PCR Protocols: A Guide to Methods and Applications) or chemical synthesis). Appropriate PCR primers can be used, for example, to introduce new functions, such as restriction cleavage sites or termination codons, into a DNA sequence according to the invention. In this way, it is possible to appropriately design sequences according to the invention for transferring them into cloning vectors.

The present invention also relates to expression vectors which contain a DNA sequence according to the invention, for example as previously disclosed or as claimed in claims 1 to 4 (claim 5). In addition to at least one DNA sequence according to the invention, such expression vectors according to the invention (for example plasmids) also contain promoter regions and terminator regions, where appropriate also at least one marker gene (for example antibiotic resistance gene) and/or at least one signal sequence for transporting the translated protein, for example into a particular cell organelle or into the extracellular space.

The present invention also relates to host cells which have been transformed with an expression vector according to the invention (claim 6). Prokaryotes, yeasts or higher eukaryotic cells are suitable host cells for cloning or expressing the DNA sequences according to the invention. “Prokaryotes” expressly includes Gram-negative or Gram-positive organisms. Those which should be mentioned here are E. coli or bacilli. The strains E. coli 294, E. coli B and E. coli X1776, and also E. coli W3110, are disclosed as being preferred host cells for cloning the DNA sequences according to the invention. The bacilli include Bacillus subtilis, Salmonella typhimurium or the like. As already mentioned above, the expression vectors typically contain at least one bacterium-specific signal sequence for transporting the protein into the culture medium. Apart from prokaryotes, eukaryotic microbes which have been transformed with an expression vector according to the invention are also suitable host cells. Thus, filamentous fungi or yeasts, for example, can be used as suitable host cells for the vectors encoding the DNA sequences according to the invention. Those which should be mentioned are, in particular, Saccharomyces cerevesiae or ordinary bakers yeast (Stinchcomb et al., Nature, 282:39, (1997)).

However, in a preferred embodiment, cells derived from multicellular organisms are chosen for expressing DNA sequences according to the invention. This also takes place in the light of a (N-coupled and/or O-coupled) glycosylation of the encoded proteins possibly being required. This function can be performed in a suitable manner in higher eukaryotic cells in contrast to prokaryotic cells. In principle, any higher eukaryotic cell culture is available as host cell, even though cells from mammals, for example monkeys, rats, hamsters or humans, are very particularly preferred. The skilled person is familiar with a large number of established cell lines. In an enumeration which is in no way conclusive, the following cell lines may be mentioned: 293T (embryonic kidney cell line), (Graham et al., J. Gen. Virol., 36:59 (1997)), BHK (baby hamster kidney cells), CHO (hamster ovary cells), (Urlaub and Chasin, P.N.A.S. (USA) 77:4216, (1980)), HeLa (human cervical carcinoma cells) and other cell lines which have been established particularly for laboratory use.

Within the meaning of the present invention, preference is given to transfecting cells of the mammalian immune system, especially the human immune system, with expression vectors containing the DNA sequences according to the invention (claim 7). The transfection of B cells and/or T cells comes particularly into consideration.

Another aspect of the present invention is represented by the gene products of the DNA sequences according to the invention (claim 8). Within the meaning of this invention, gene products are understood as being both primary transcripts, that is RNA, preferably mRNA, and proteins or polypeptides, in particular in purified form (claim 9). According to the invention, these proteins possess at least one BHNo domain and regulate or transport, in particular, apoptotic, where appropriate also inflammatory, signals. A purified gene product is preferred when it contains the amino acid sequence (for a BHNo domain) specified in FIG. 7 or a functionally homologous allele, fragment or derivative of this sequence (claim 10). In this connection, a derivative is understood as meaning, in particular, those AA sequences which have been altered by their side chains having been modified. For example, by conjugating an antibody, enzyme or receptor to an AA sequence according to the invention. However, derivatives can also [lacuna] the coupling of a sugar or fat (fatty acid) residue or of a phosphate group or of any arbitrary modification of a side chain, in particular of a free OH group or NH2 group or at the N terminus or C terminus. In addition to this, the term “derivative” also includes fusion proteins, in which a DNA sequence according to the invention is consequently coupled to any arbitrary oligopeptides or polypeptides.

Sequences which are characterized by at least one AA change (insertion, substitution) as compared with the native sequence are termed “analogs”. Preference is given to conservative substitutions in which the physicochemical character of the AA which has been replaced is preserved (polar AA, long aliphatic chain, short aliphatic chain, negatively or positively charged AA, AA possessing an aromatic group). Preference is given to analogs which retain the secondary structure which was present in the native sequence. In addition to conservative substitutions, it is also possible, according to the invention, to introduce AA variations which are less conservative into the native sequence. In connection with this, they typically retain their function as transducers of an apoptotic signal. The effect of a substitution or deletion can be readily examined by means of appropriate analyses, binding assays or cytotoxic tests.

Nevertheless, the invention also includes sequences which elicited what is termed a dominant-negative effect, i.e. which, as a result of their altered primary sequence, still exhibit binding activity toward a sequence which is located upstream in the cascade but are unable to transmit the signal downstream. These analogs therefore function as inhibitors of apoptosis. Such analogs are prepared by recombinant DNA procedures, typically by subjecting a DNA sequence which encodes a protein according to the invention, containing a BHNo domain (typically Bcl-rambo), to what is termed site-directed mutagenesis. This thereby produces the DNA sequence which underlies the analog and which finally expresses the protein in a recombinant cell culture Sambrook et al., 1989, see above). The invention also discloses all the derivatives of the above-described analogs, in exactly the same way as the DNA sequences underlying the above-described AA sequences.

Furthermore, fragments of a native AA sequence according to the invention also form part of the subject matter of the present invention. Fragments are characterized by deletions (N-terminal or C-terminal deletions or else intrasequence deletions). The fragments can have a dominant-negative or dominant-positive effect.

However, the gene products (proteins) according to the invention also include all those gene products (proteins) which, according to the invention, are derived, after transcription and translation, from DNA derivatives, DNA fragments or DNA alleles of the DNA sequence specified in FIG. 9.

In addition to this, the proteins according to the invention can be chemically modified. Thus, for example, a protecting group can be present at the N terminus. Glycosyl groups can be attached to hydroxyl or amino groups, lipids can be covalently linked to the protein according to the invention, as can phosphates or acetyl groups and the like. Any arbitrary chemical substances, compounds or groups can also be bonded, by any arbitrary synthetic pathway, to the protein according to the invention. It is also possible for additional amino acids, for example in the form of individual amino acids or in the form of peptides or in the form of protein domains and the like, to be fused to the N terminus and/or C terminus of a protein according to the invention, in particular at the N terminus or C terminus of a BHNo domain, where appropriate, however, also integrated into the sequence of the BHNo domain according to the invention. Particular preference is given, in this connection, to what are termed signal sequences or leader sequences at the N terminus of the amino acid sequence according to the invention, which sequences lead the peptide cotranslationally or posttranslationally into a particular cell organelle or into the extracellular space (or the culture medium). It is also possible for amino acid sequences which, as antigens, permit the binding of the amino acid sequence according to the invention to antibodies, to be present at the N terminus or at the C terminus. Mention should be made here, in particular, of the flag peptide, whose sequence in the single-letter amino acid code is: DYKDDDDK. Or else a His tag containing at least 3, preferably at least 6, histidine residues. These sequences possess powerfully antigenic properties and consequently enable the recombinant protein to be rapidly examined and readily purified. Monoclonal antibodies which bind the flag peptide can be obtained from Eastman Kodak Co., Scientific Imaging Systems Division, New Haven, Conn.

Genomic DNA sequences according to the invention can be laid down, on the strand of the genetic information molecule, in numerous exons which are separated from each other by introns, for example as shown in FIG. 1C. As a result, DNA sequences according to the invention can be present as cDNA (without any intron sequences) or genomically, containing at least one intron sequence. As shown in FIG. 1C, taking the human Bcl-rambo sequence as an example, DNA sequences according to the invention can contain one, two, three, four or 5 intron sequences (in any arbitrary combination, for example only intron 2 (5.7 kb) and intron 3 (7.2 kb)) or else be present as cDNA (without any intron sequence). All conceivable SPLICE variants (at the mRNA level) are also included, as gene products, in the subject matter of the invention. The present invention also covers all the proteins which are encoded by these splice variants which differ at the mRNA level. As a result, proteins according to the invention can possess the BHNo domain with or without at least one further exon sequence or part of an exon sequence, for example the AA sequence of exon III and exon IV and the first 15 AA of exon V in combination with the amino acid sequence of the BHNo domain on exon VI (with or without a transmembrane domain).

The present invention also relates to an antibody which recognizes an epitope on a gene product according to the invention, in particular a protein according to the invention (claim 11). Within the meaning of the present invention, the term “antibody” encompasses both polyclonal antibodies and monoclonal antibodies (claim 12), chimeric antibodies and anti-idiotypic antibodies (directed against antibodies according to the invention), all of which can be present in bound or soluble form and, where appropriate, labeled with labels, as well as fragments of the abovementioned antibodies. In addition to the fragments of antibodies according to the invention on their own, antibodies according to the invention can also be present in recombinant form, as fusion proteins containing other (protein) constituents. Fragments as such, or fragments of antibodies according to the invention as constituents of fusion proteins, are typically prepared by the methods of enzymic cleavage or protein synthesis or by the recombination methods with which the skilled person is familiar.

The polyclonal antibodies are heterogeneous mixtures of antibody molecules which are prepared from the sera of animals which have been immunized with an antigen. However, the invention also relates to polyclonal monospecific antibodies which are obtained after purifying the antibodies (for example through a column which are loaded with peptides of a specific epitope). A monoclonal antibody contains an essentially homogeneous population of antibodies which are directed specifically against antigens, with the antibodies exhibiting essentially identical epitope-binding sites. Monoclonal antibodies can be obtained by the methods which are known in the prior art (e.g. Köhler and Milstein, Nature, 256, 495-397, (1975); U.S. Pat. No. 4,376,110; Ausubel et al., Harlow and Lane “Antibodies”: Laboratory Manual, Cold Spring, Harbor Laboratory (1988)). The description contained in the abovementioned literature references is hereby incorporated, as a constituent of the present invention, into the disclosure of the present invention. Antibodies according to the invention can belong to one of the following immunoglobulin classes: IgG, IgM, IgE, IgA and GILD and, where appropriate, a subclass of the previously mentioned classes. A hybridoma cell clone which produces monoclonal antibodies according to the invention can be cultured in vitro, in situ or in vivo. Large titers of monoclonal antibodies are preferably prepared in vivo or in situ.

The chimeric antibodies according to the invention are molecules which contain different constituents which are derived from different animal species (e.g. antibodies which possess a variable region which is derived from a mouse monoclonal antibody and a constant region belonging to a human immunoglobulin). Chimeric antibodies are preferably employed to reduce immunogenicity during use, on the one hand, and, on the other hand, to increase the yields during production, for example while murine monoclonal antibodies give higher yields from hybridoma cell lines, they also lead to higher immunogenicity in humans, which means that preference is given to using human/murine chimeric antibodies. Chimeric antibodies, and methods for preparing them, are disclosed in the prior art (Cabilly et al., Proc. Natl. Sci. USA 81: 3273-3277 (1984); Morrison et al. Proc. Natl. Acad. Sci USA 81:6851-6855 (1984); Boulianne et al. Nature 312 643-646 (1984); Cabilly et al., EP-A-125023; Neuberger et al., Nature 314: 268-270 (1985); Taniguchi et al., EP-A-171496; Morrison et al., EP-A-173494; Neuberger et al., WO 86/01533; Kudo et al., EP-A-184187; Sahagan et al., J. Immunol. 137: 1066-1074 (1986); Robinson et al., WO 87/02671; Liu et al., Proc. Natl. Acad. Sci USA 84:3439-3443 (1987); Sun et al., Proc. Natl. Acad. Sci USA 84:214218 (1987); Better et al., Science 240: 1041-1043 (1988) and Harlow and Lane, Antibodies: A Laboratory Manual, as cited above. These literature references are hereby incorporated, as belonging to the disclosure, into the present invention.

Very particular preference is given to an antibody according to the invention which is directed against a sequence segment, as epitope, on the BHNo domain (claim 13).

An antiidiotypic antibody according to the invention is an antibody which recognizes a determinant which is generally associated with the antigen-binding site of an antibody according to the invention. An antiidiotypic antibody can be prepared by immunizing an animal of the same species and the same genetic type (e.g. a mouse strain) as the starting point for a monoclonal antibody against which an antiidiotypic antibody according to the invention is directed. The immunized animal will recognize the idiotypic determinants of the immunizing antibody by producing an antibody (namely an antiidiotypic antibody according to the invention) which is directed against the idiotypic determinants (U.S. Pat. No. 4,699,880). An antiidiotypic antibody according to the invention can also be used as an immunogen in order to illicit an immune response in another animal and in order, in this animal, to lead to the production of what is termed an anti-antiidiotypic antibody. The anti-antiidiotypic antibody can, but does not have to, be identical, as regards its epitope construction, to the original monoclonal antibody which elicited the antiidiotypic reaction. In this way, it is possible, by using antibodies which are directed against idiotypic determinants of a monoclonal antibody, to identify other clones which express antibodies of identical specificity.

Monoclonal antibodies which are directed against proteins according to the invention or analogs, fragments or derivatives of these proteins according to the invention can be used in order to induce the binding of antiidiotypic antibodies in appropriate animals, for example the BALB/c mouse. Cells from the spleen of such an immunized mouse can be used to produce antiidiotypic hybridoma cell lines which secrete antiidiotypic monoclonal antibodies. It is furthermore possible to couple antiidiotypic monoclonal antibodies to a support (KLH, keyhole limpet hemocyanin) and then to use them to immunize other BALB/c mice. The sera of these mice then contain anti-antiidiotypic antibodies which have the binding properties of the original monoclonal antibodies and are specific for an epitope of the protein according to the invention or of a fragment or derivative thereof. This way, the antiidiotypic monoclonal antibodies have their own idiotypic epitopes or “idiotopes” which are structurally similar to the epitope to be investigated.

The term “antibody” is intended to include both intact molecules and fragments of these molecules, for example Fab and F(ab′)₂. Fab and F(ab′)₂ fragments lack an Fc fragment, as is present, for example, in an intact antibody, which means that they can be transported more rapidly in the blood circulation and exhibit comparatively less nonspecific tissue binding than do intact antibodies. In this connection, it is stressed that Fab and F(ab′)₂ fragments of antibodies according to the invention can be used in the detection and quantification of proteins according to the invention. These fragments are typically prepared by proteolytic cleavage, by using enzymes such as papain (for preparing Fab fragments) or pepsin (for preparing F(ab′)₂ fragments).

Antibodies according to the invention, including the fragments of these antibodies, can be used for quantitatively or qualitatively detecting protein according to the invention in a sample or else for detecting cells which are expressing and, where appropriate, secreting the proteins according to the invention. The detection can be affected using immunofluorescence methods which are carried out [lacuna] fluorescence-labeled antibodies in combination with light microscopy, flow cytometry or fluorometric detection.

Antibodies according to the invention (or fragments of these antibodies) are suitable for histological investigations, for example in the context of immunofluorescence or immunoelectromicroscopy, for detecting a protein according to the invention in situ. The in-situ detection can be effected by a histological sample being taken from a patient and labeled antibodies according to the invention being added to such a sample. The antibody (or a fragment of this antibody) is applied in labeled form to the biological sample. In this way, it is not only possible to determine the presence of protein according to the invention in the sample but also to determine the distribution of the protein according to the invention in the tissue being investigated. The biological sample can be a biological liquid, a tissue extract, harvested cells, such as immune cells or cardiac muscle cells or liver cells, or, in a general manner, the biological sample can be cells which have been incubated in a tissue culture. The labeled antibody can be detected using methods which are known in the prior art and which depend on the nature of the label (e.g. using fluorescence methods). However, the biological sample can also be applied to a solid phase support, such as nitrocellulose or another support material, such that the cells, cell parts or soluble proteins are immobilized. The support can be washed once or more than once with a suitable buffer and subsequently treated with a detectably labeled antibody in accordance with the present invention. The solid phase support can then be washed for a second time with the buffer in order to remove unbound antibodies. A conventional method can then be used to determine the quantity of bound label on the solid phase support.

Suitable supports are, in particular, glass, polystyrene, polypropylene, polyethylene, dextran, nylon amylases, natural or modified celluloses, polyacrylamides and magnetite. In order to fulfill the conditions in accordance with the present invention, the support can either be soluble under certain conditions or be insoluble. The support material can assume any arbitrary forms, for example be in the form of beads or be cylindrical or spherical, with polystyrene beads being preferred as the support.

An antibody can be labeled in a detectable manner in a variety of ways. For example, the antibody can be bound to an enzyme, with it finally being possible to use the enzyme in an immunoassay (EIA). The enzyme can then subsequently react with an appropriate substrate, leading to the formation of a chemical compound which can be detected and, where appropriate, quantified in a manner with which the skilled person is familiar, for example by means of spectrophotometry, fluorometry or other optical methods. The enzyme can be malate dehydrogenase, staphylococcal nuclease, delta-5-steroid isomerase, yeast alcohol dehydrogenase, alphaglycerophosphate dehydrogenase, triose phosphate isomerase, horse radish peroxidase, alkali phosphatase, aspariginase, glucose oxidase, beta-galactosidase, ribonuclease, urease, catalase, glucose-6-phosphate dehydrogenase, glucoamylase or acetylcholine esterase. Detection is then made possible using a chromogenic substrate which is specific for the enzyme used for the label and can finally be effected, for example, by visual comparison of the substrate transformed by the enzyme reaction and the control standard.

The detection can also be effected using other immunoassays, for example by means of radioactively labeling the antibodies or antibody fragments (that is by using a radioimmunoassay (RIA; Laboratory Techniques and Biochemistry in Molecular Biology, Work, T. et al. North Holland Publishing Company, New York (1978). In this method, the radioactive isotope can be detected and quantified by using scintillation counters or by means of autoradiography.

Fluorescent compounds, for example compounds such as fluorescine isothiocyanate, rhodamine, phyoerythrin, phycocyanin, allophycocyanin, o-phthaldehyde and fluorescamine, can also be used for the labeling. It is also possible to use fluorescence-emitting metals, such as ¹⁵²E or other metals from the lanthanide group. These metals are coupled to the antibody by way of chelate groups such as diethylenetriaminepentaacetic acid (ETPA) or EDTA. Furthermore, the antibody according to the invention can be coupled by way of a compound which acts with the aid of chemiluminescence. The presence of the chemiluminescence-labeled antibody is then detected by way of the luminescence which arises during the course of a chemical reaction. Examples of such compounds are luminol, isoluminol, acridinium ester, imidazole, acridinium salt and oxalate ester. It is also equally possible to use bioluminescent compounds. Bioluminescence is a subvariety of chemiluminescence which is found in biological systems, with a catalytic protein enhancing the efficiency of the chemiluminescent reaction. The bioluminescent protein is once again detected by way of the luminescence, with suitable bioluminescent compounds being, for example, luciferin, luciferase and aequorin.

An antibody according to the invention can come to be used in an immunometric assay, also known as a two-site or sandwich assay. Typical immunometric assay systems include what are termed “forward” assays, which are characterized in that antibodies according to the invention are bound to a solid phase system and in that the antibody is in this way brought into contact with the sample which is being investigated. In this way, the antigen in the sample is isolated from the sample by forming a binary solid-phase antibody/antigen complex. After a suitable incubation time, the solid support is washed in order to remove the remainder of the liquid sample, including any unbound antigen, and, after that, brought into contact with a solution which contains an unknown quantity of labeled detected antibody. In this context, the labeled antibody serves as what is termed a reporter molecule. After a second incubation time, which enables the labeled antibody to associate with the antigen which is bound to the solid phase, the solid phase carrier is once again washed in order to remove labeled antibodies which have not reacted.

In an alternative assay form, it is also possible to use what is termed a sandwich assay. In this assay, a single incubation step can be sufficient if the antibody which is bound to the solid phase and the labeled antibody are both applied simultaneously to the sample to be tested. After the incubation has been concluded, the solid phase support is washed in order to remove residues of the liquid sample and the unassociated labeled antibodies. The presence of labeled antibody on the solid phase support is determined in precisely the same way as in the case of the conventional “forward” sandwich assay. In what is termed the reverse assay, a solution of the labeled antibody is first of all added to the liquid sample with this being followed, stepwise, by the admixing of unlabeled antibody, which is bound to a solid phase support, after a suitable incubation time has elapsed. After a second incubation step, the solid phase support is washed in a conventional manner in order to free it from sample residues and from labeled antibody which has not reacted. The labeled antibody which has reacted with the solid phase support is then determined as described above.

Another aspect of the present invention is a method for isolating gene products which contain at least one amino acid sequence according to the invention, in particular a BHNo domain (as depicted in FIG. 7), with the host cells being transformed with an expression vector according to the invention and then being cultured under suitable, expression-promoting conditions such that the gene product can finally be purified from the culture (claim 14). In this connection, the protein corresponding to the DNA sequence according to the invention can, depending on the expression system, be isolated from a culture medium or from cell extracts. The skilled person can readily perceive that the particular isolation methods, and the method employed when purifying the recombinant protein which is encoded by a DNA according to the invention are highly dependent on the host cell type or else on whether the protein is secreted into the medium. For example, it is possible to use expression systems which lead to the recombinant protein being secreted out of the host cell. In this case, it is necessary to use commercially available protein concentration filters, for example Amicon or Millipore Pelicon, to concentrate the culture medium. Following the concentration step, a purification step, for example a gel filtration step or column-chromatographic methods, can then take place. However, as an alternative, it is also possible to use an anion exchanger which possesses a matrix containing DEAE.

Any materials which are known from protein purification, for example acrylamide or agarose or dextran or the like, can be used as the matrix in this connection. However, it is also possible to use a cation exchanger, which then typically contains carboxymethyl groups. HPLC steps can then be used for the further purification of a polypeptide which is encoded by a DNA according to the invention. The HPLC purification can consist of one or more steps. The reversed-phase method is used, in particular. These steps serve to obtain a recombinant protein which is derived from a DNA sequence according to the invention and which is essentially homogeneous.

In addition to bacterial cell cultures, it is also possible to use transformed yeast cells for isolating the gene product. In this case, the translated protein may be secreted, thereby simplifying the protein purification. Recombinant protein which is secreted from a yeast host cell can be obtained using methods which are disclosed in Urdal et al. (J. Chromato. 296:171 (1994)).

Another aspect of the present invention is a method for expressing gene products which contain at least one BHNo domain, including all the derivatives, analogs and fragments, with host cells being transformed, in this method, with an expression vector which contains a DNA sequence according to the invention (claim 15). This method for expressing gene products which are based on a DNA sequence according to the invention is not employed to concentrate and purify the corresponding gene product but, on the contrary, to use the introduction of the DNA sequences according to the invention to exert an influence on cell metabolism by way of expressing the pertinent gene product. The thought behind this is, in particular, that of using the host cells which have been transformed with expression vectors as pharmaceuticals or for producing a pharmaceutical, in particular for the purpose of treating diseases which [lacuna] on incorrectly regulated intracellular signal transduction or incorrectly regulated homeostasis of survival and death signals, especially for the purpose of regulating apoptosis (claim 16). The autologous or allogenic host cells which have in this way been transformed ex vivo in accordance with the invention can then be transplanted into patients.

Depending on the conditions desired, a DNA sequence according to the invention can be linked behind a constitutively activated promoter, or, on the contrary, can be linked operably to a promoter which can be induced as desired, on the expression vector which is used for the cell transfection. When the promoter is inducible, the DNA sequence according to the invention will preferably be able to be employed as a suicide gene. This means that, following transfection of the host cell, the transcription of the cell death-inducing sequence according to the invention is, for example, initially repressed or unable to be activated. The host cells, where appropriate transfected with at least one further gene or gene fragment (on the same or on at least one further expression vector), for example a tumor repressor gene or a therapeutic gene in the case of hereditary diseases, are then (re)transplanted into the patient for the purpose of gene therapy (autogenous or allogenic host cells). If necessary, depending on the medical indication, the transcription of the apoptosis-inducing DNA according to the invention can, after transplantation, be initiated, for the purpose of inducing suicide of the cell, by administering an inducer or negative repressor. In this way, depending on the course of the gene therapy, the transplanted host cell can be kept alive or fall victim to the apoptosis which is induced selectively. In a quite general manner, transfection of host cells with an inducible DNA sequence according to the invention is therefore suitable for negatively selecting appropriately transfected host cells.

In addition, the invention also discloses doubly (dominant)-negative mutants (DN), which are able to suppress cell death which may optionally be induced extracellularly. For example, these mutants may be fragments of sequences according to the invention which, while being able to receive the apoptotic signal, are no longer able to transmit it onwards in a cascade-like manner. Such DN sequences according to the invention are suitable, after appropriate transfection of host cells, for protecting against apoptosis, or at least against particular apoptotic events, and rendering immortal cells which have been transfected with them. Uses of such host cells, which are apoptosis-immune or partially apoptosis-immune, arise, for example, within the context of gene therapy in connection with diseases which are etiologically induced by the incorrectly regulated destruction of cells (or tissues), are at least accompanied by pathological cell destruction, or exhibit the pathological death of large numbers of cells as a symptom (Parkinson's disease, Alzheimer's disease or viral diseases, for example HIV infections). Depending on the indication, the host cells are either transfected solely with DN mutants according to the invention or else at least cotransfected with these mutants, i.e. also transfected with at least one further therapeutic gene on the same vector or on at least one further vector. In a quite general manner, the transfection of host cells with a DN DNA sequence according to the invention is therefore suitable for positively selecting appropriately transfected host cells.

Resulting from the abovementioned methods, the host cell lines become resistant toward a large number of apoptotic stimulants. The cells which are manipulated in vitro using the expression vectors according to the invention are preferably those cells which, in the body, have fallen victim to faulty regulation of apoptosis and can now be regenerated, following transplantation, with transformed cells according to the invention. In particular, use is made, in such a gene-therapy approach, of sequences according to the invention which, as doubly negative mutants, block apoptotic events.

However, the present inventive concept also includes a gene-therapy method which can be carried out in vivo. Use is made, for this purpose, of vectors (e.g. liposomes, adenoviruses, retroviruses or similar, or else naked DNA) which insert the DNA sequences according to the invention selectively into the desired target cells in the body. The target cells are typically cells whose death/survival homeostasis is disturbed, in particular cells which pathologically exhibit an increased disposition for apoptosis (for example in the case of diabetes mellitus, Parkinson's disease and autoimmune diseases). In this connection, it is possible to use fragments of a DNA sequence according to the invention which display an inhibitory effect, for example DN DNA sequences which are essentially no longer able to transmit biological signals, that is which no longer exhibit any functional biological or apoptosis-transducing ability.

However, vectors which are able to induce apoptosis by expressing sequences according to the invention also come into consideration as pharmaceuticals in in-vivo gene-therapy methods. In particular, the invention discloses the use of such viral or liposomal vectors for treating tumor diseases. However, naked DNA according to the invention can also be employed as a pharmaceutical in this regard.

The present invention also relates to the use of a DNA sequence (alleles, derivatives or fragments) according to the invention, or of a gene product according to the invention, for treating diseases which are due to incorrectly regulated intracellular signal transduction (claim 16). This use, according to the invention, of DNA sequences also includes using above-described expression vectors according to the invention which contain a nucleotide sequence according to the invention for example a nucleotide sequence which is disclosed in FIG. 9 or a functional derivative, fragment, analog or allele of such a sequence (or else a nonfunctional derivative of such a sequence, for example a DN mutant) with the aim of correcting the incorrectly controlled intracellular signal transduction. The use can be affected in accordance with gene-therapy methods, by means of injecting naked DNA according to the invention or the protein, or by using gene transporters, in particular viral or liposomal vectors. The present invention therefore also relates both to the use of such DNA sequences, gene products and expression vectors according to the invention, and to the use of cells according to the invention which are transfected with expression vectors according to the invention, as pharmaceuticals.

Polypeptides, expression vectors or DNA sequences according to the invention, or their respective derivatives, analogs or fragments, can serve various purposes, for example enhance the apoptotic reaction when such an effect is desired, for example in the case of antitumor, antiinflammatory or anti-HIV applications. In this connection, preference is given to using a DNA sequence according to the invention or a gene product according to the invention when the disease is one which is associated with incorrectly regulated cell apoptosis, in particular a deficiency in the apoptosis of particular cells in a multicellular organism (claim 17). Very particular preference is given to using such a DNA sequence or such a gene product for treating (for producing a pharmaceutical for treating) tumor diseases or else for controlling an overshooting immune response in the case of autoimmune diseases (claim 18). Mention may be made, in particular, of the treatment of solid tumors, for example of tumors in epithelial tissue (for example intestine and lung), brain tumors (for example glioblastoma and astrocytoma), sarcomas and tumors of the immune system, and also leukemias, in particular acute myeloid or chronic myeloid leukemia.

In order to insert the proteins or DNA sequences according to the invention into the patient cells to be treated, it. is possible, for example, to use a recombinant animal virus (for example derived from vaccinia), with at least two genes being added. In the first place, a gene for a ligand which binds to a receptor on the target cell (for example gp120 in the case of CD4-carrying lymphocytes) and, in second place, the DNA sequence according to the invention, for inducing or augmenting cell death.

On the other hand, it is also possible to use, as pharmaceuticals, derivatives or fragments of sequences according to the invention which are nonfunctional as regards transmitting the apoptotic signal, for example DN mutants of sequences according to the invention. They serve, in particular, for treating degenerative diseases, for example neurodegenerative diseases, autoimmune diseases or infectious diseases. The invention therefore also makes available compounds which are characterized in that they modulate, in particular inhibit, the function of the gene products (proteins) according to the invention as intracellular signal molecule of an apoptotic signal cascade for inducing cell death (claim 19). In particular, compounds according to the invention block the specific, Bcl-rambo-mediated activation of caspase-3 (claim 20). A chemical compound according to the invention which blocks apoptosis is preferably an oligopeptide or polypeptide (see above with regard to DN mutant) which can either be chemically modified (for example for facilitating passage through the cell membrane, in particular by means of terminally located (especially N-terminal) sequence regions) or else not modified. Where appropriate, such an oligopeptide or polypeptide can also be chemically modified such that the amide-like bond between the individual amino acids is replaced with an alternative chemical group (for example sulfur bridge or phosphorus bridge) which is resistant to proteolytic breakdown.

An inhibition of apoptosis, as is desired, for example, in the case of septic shock, GvHD or acute hepatitis, can also be achieved by using methods which are familiar to the skilled person to insert oligonucleotides which encode an antisense strand of the native Bcl-rambo sequence, or of another native protein containing a BHNo domain, into the cells concerned. This thereby blocks translation of the native mRNA of Bcl-rambo, for example, in the cells which have been transformed in this way, thereby increasing, as has been shown, the ability of the transfected cell to survive. In this case, too, it is possible to carry out this method using recombinant viruses. The cell apoptosis which may be pathologically increased in the case of diseases which are due to corresponding faulty regulation can also be treated by means of ribozyme methods. Ribozymes which are able to cut a target mRNA are used for this purpose. In the present case, the invention therefore discloses ribozymes which are able to cleave native Bcl-rambo mRNA or the mRNA for other BHNo-domain-containing proteins. In this connection, ribozymes according to the invention must be able to interact with the target mRNA according to the invention, for example by way of base-pairing, and then cleave the mRNA, in order to block the translation of Bcl-rambo, for example. The ribozymes according to the invention are inserted into the target cells using suitable vectors (in particular plasmids or modified animal viruses, in particular retroviruses), with the vectors containing a cDNA sequence for a ribozyme according to the invention together with other sequences, where appropriate.

A chemical compound according to the invention is preferably an organochemical compound having a molecular weight of <5000, in particular <3000, especially less than <1500 and is typically well tolerated physiologically and can preferably pass through the blood/brain barrier (claim 21). Where appropriate, it will be a constituent of a composition containing at least one further active compound and also, preferably, auxiliary substances and/or additives and will be able to be used as a pharmaceutical. The organic molecule will be particularly preferred when the binding constant for binding to a protein according to the invention, in particular to the BHNo domain of a protein according to the invention, is at least 10⁷ mol⁻¹. The compound according to the invention will preferably be so constituted that it is able to pass through the cell membrane, whether by diffusion or using (intra)membrane transport proteins (claim 22), optionally after appropriate modification, for example with an AA sequence which has been coupled on.

In another preferred embodiment of the present invention, the compound according to the invention is an antibody, preferably an antibody which is directed against the BHNo domain of a protein according to the invention, which is inserted into retransplanted host cells ex vivo, or inserted into host cells using gene-therapy in-vivo methods, and, as an “intrabody” in the cells, is not secreted but can instead exert its effect intracellularly. Such “intrabodies” according to the invention protect the cells from an apoptotic reaction. Such an approach will typically be suitable for cells of those tissues which, in the patient, exhibit a pathophysiologically exaggerated apoptotic behavior, that is, for example, in the case of pancreatic cells, keratinocytes, connective tissue cells, immune cells, neurons or muscle cells. In addition to the antibodies or intrabodies, cells which have been modified gene-therapeutically with “intrabodies” according to the invention in this way also form part of the subject matter of the present invention.

In accordance with the invention, it has been observed that members of what is termed the IAP substance class, in particular, act as inhibitors of Bcl-rambo-mediated apoptosis. In addition to c-IAP-1 and c-IAP2, the xIAPs are to be singled out, in particular, as being strong inhibitors. In this connection, the entire content of the document by Deveraux and Reed ((1999), Genes Dev. 13(3), 239-252) is hereby explicitly incorporated by reference into the disclosure of the present inventive subject matter.

A chemical compound according to the invention which possesses the function of blockading the apoptotic function, for example, of physiological proteins according to the invention can be used as a pharmaceutical. In particular, a chemical compound according to the invention, for producing a pharmaceutical, is suitable for treating diseases for which a pathological hyperapoptotic reaction is at least partially causal or symptomatic. In this way, an inhibitor according to the invention (for example an antibody having an inhibitory effect (for example an intrabody), a ribozyme, antisense RNA, dominantly negative mutants, or one of the abovementioned inhibitory organic compounds) of the cellular function of a protein according to the invention, that is, for example, of the apoptotic reaction, can be used as a pharmaceutical and, very particularly, in the treatment of the following diseases, or for producing a pharmaceutical for treating the following diseases: autoimmune diseases, in particular diabetes, psoriasis, multiple sclerosis, rheumatoid arthritis or asthma, viral infectious diseases (for example HIV), degenerative diseases, in particular neurodegenerative diseases, for example Alzheimer's disease or Parkinson's disease, or muscular dystrophies, GvHD (for example liver, kidney or heart) or acute hepatitis (claim 25). The abovementioned apoptosis-inhibiting substances according to the invention can also be a component of a pharmaceutical composition which can contain further pharmaceutical carrier substances, auxiliary substances and/or additives in order, for example, to stabilize such compositions for therapeutic administration or improve the biological availability and/or pharmacodynamics.

The present invention also relates to methods (screening methods) for identifying compounds which possess inhibitory properties in regard to the induction or transmission of signals which are connected with apoptotic reactions which are elicited by physiologically occurring sequences according to the invention. Methods according to the invention provide for (a) transfecting cells with an expression vector as claimed in claim 5, in particular an expression vector which encodes the Bcl-rambo protein, where appropriate at least one expression vector which encodes at least one apoptosis inhibitor and, where appropriate, at least one expression vector which encodes at least one reporter gene, and (b) measuring, for the host cell system obtained in accordance with (a), a parameter which is suitable for observing the Bcl-rambo-mediated apoptosis, in particular the activation of caspase-3, which occurs after adding a test compound as compared with the control without any addition of a test compound (claim 23). In this regard, preference is given, in accordance with the method according to the invention, to setting up several parallel experiments containing increasing concentrations of the test substance in order to be able to determine its ID₅₀ value if it exhibits an apoptosis-inhibiting effect. Preference is given to a screening method according to the invention when the apoptosis inhibitor employed for the transfection is FLIP, CrmA, DN-FADD and/or DN-caspase-9 and the reporter gene is, for example, β-galactosidase (claim 24).

A screening method according to the invention can also be carried out using what are termed proteomics techniques. For this, typical differences in the expression pattern shown by cells exhibiting an apoptotic reaction and control cells are recorded experimentally for the purpose of determining a standard. 2D gel electrophoresis is the method which is typically used in this instance.

Sequences according to the invention can also be used in methods whose aim is to identify cellular interaction partners of the Bcl-rambo protein. Such a method can be carried out, for example, using what is termed the yeast two-hybrid method, with which the skilled person is familiar (claim 26) or using affinity chromatography methods. This thereby discloses the use of a DNA sequence according to the invention, or of a gene product of such a sequence according to the invention, for identifying other proteins which are involved in apoptotic signal transduction (claim 27). In the affinity chromatography, AA sequences according to the invention, or their derivatives, analogs, fragments or alleles, are coupled to a matrix, brought into contact with cell extracts, for example, and, after a washing step, elution takes place, with this being followed by characterization of the eluted complexes.

The present invention is explained in more detail by means of the following figures:

FIG. 1 represents a comparison of the sequences of the Bcl-rambo protein according to the invention with antiapoptotic proteins of the Bcl-2 family. These include the sequences of the human Bcl-2 protein, of the human Bcl-XL protein and of the human Bcl-w protein. Just like these proteins of the Bcl-2 family, the human Bcl-rambo sequence according to the invention also exhibits the characteristic sequence segments BH1, BH2, BH3 and BH4 as well as a transmembrane region (MA). The abovementioned sequence segments are boxed in FIG. 1. Amino acids which are identical in the four sequences have a black background while amino acids which are similar have a gray background. It is clear from FIG. 1a that, in the case of the human Bcl-rambo sequence, a sequence segment comprising more than 200 amino acids, followed by a C-terminal anchor region, is inserted C-terminally of the BH2 region. This segment, which is termed, according to the invention, the BHNo domain, contains so-called repeats (repeat A and repeat B) which, as shown in FIG. 1B, in each case exhibit at least 80% identity. FIG. 1B compares the sequences of the two regions designated “repeat A”, and of the two regions designated “repeat B”, which occur in the C-terminal region of Bcl-rambo.

FIG. 1C shows the exon/intron organization of the human Bcl-rambo gene. The gene comprises 6 exons (numbered consecutively from I to VI, in each case separated by intron sequences). ExonI is separated by 27.4 kb from ExonII, ExonII is separated by 5.7 kb from ExonIII, ExonIII is separated by 7.4 kb from ExonIV, ExonIV is separated by 6 kb from ExonV and, finally, ExonV is separated from ExonVI by 24.3 kb. In addition, FIG. 1c shows the positions of the BH1, BH2, BH3 and BH4 regions on the corresponding exons. The amino acid sequence segment which, according to the invention, is designated the BHNo domain is located on ExonVI between the C-terminal region of the BH2 motif and the transmembrane region. The noncoding regions, which are delimited from the coding regions by the start codon ATG and the stop codon TAG (positions indicated by arrows), are shown with a gray background.

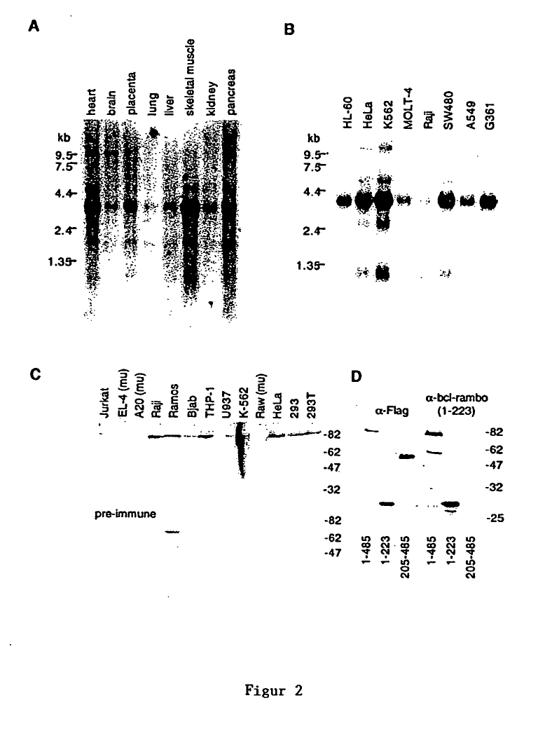

FIG. 2A shows Northern blots of different human tissues. These were exposed to ³²P-labeled cDNA fragments from the N-terminal part of Bcl-rambo. The tissues are heart, brain, placenta, lung, liver, skeletal muscle, kidney and pancreas tissue. This Northern blot shows that all the tissues investigated contain a Bcl-rambo transcript of 4.1 kb in length. The highest values for mRNA expression were determined to be in heart tissue, pancreas tissue and placenta tissue. Two markedly weaker bands of 2.1 kb and 1.2 kb in length correspond to alternative splicing forms of human Bcl-rambo. Analyses such as those shown in FIG. 2A were carried out in the case of various tumor cell lines, namely HL60, Hela, K562, MOLT-4, Raji, SW480, A549 and G361. The 4.1 kb transcript (that is, the Bcl-rambo mRNA) was detected in all the cell lines.

The Western blot analyses, which show the expression of Bcl-rambo protein in different human and murine (abbreviated to mu) cells, are in agreement with this. Bands for an 85 kD protein, which was detected using an affinity-purified antibody (AL167) which is directed against the Bcl-rambo region of homology with Bcl-2 (amino acids 1-223), are found in all the human cell lines investigated. Specifically, Western blots were analyzed for Jurkat cells, murine EL4 cells (leukemia T cells), A20 cells, Raji cells, Ramos cells, Bjab cells, Raw cells (lymphoma B cells), THP-1 cells, U937 cells, K562 cells (monocyte cell lines), HeLa cells (cervical carcinoma cells), HEK293 cells and 293 T cells (embryonic kidney cell lines).

In order to check whether the 85 kD bands in the Western blots shown in FIG. 2C correspond to the Bcl-rambo protein, 293T cells were transfected with various Flag-labeled Bcl-rambo expression plasmids. In this experiment, use was made of expression plasmids which express (a) the full-length sequence of human Bcl-rambo (amino acids 1-485), (b) the domain of homology with Bcl-2 (amino acids 1-223 of the human Bcl-rambo protein), and (c) finally, the C-terminal region of Bcl-rambo, including the BHNo domain (amino acids 205-485 of the human Bcl-rambo protein). These cell extracts were analyzed using anti-Flag antibody M2 as shown in the left-hand blot in FIG. 2D, and treated with polyclonal antibody directed against Bcl-rambo (as shown in the right-hand blot). In this experiment, it was found that the full-length human Bcl-rambo protein has a molecular weight of approx. 85 kD, that the Bcl-rambo N-terminal domain of homology with Bcl-2 is detected at approx. 28 kD and that the characteristic C-terminal domain (AA 205-485) has a surprisingly high molecular weight of approximately 52 kD.

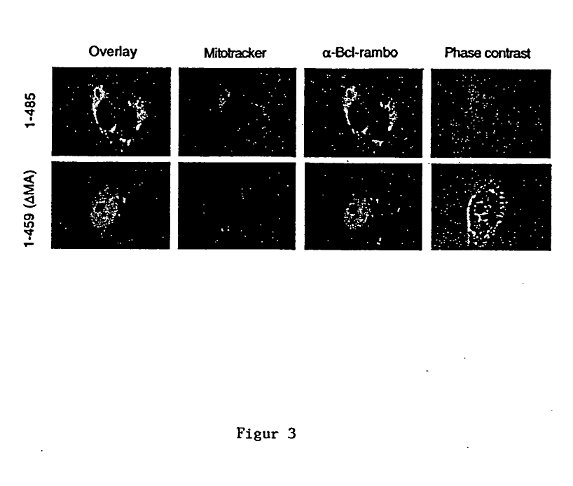

FIG. 3 shows the subcellular location of the Bcl-rambo protein, as determined using confocal laser microscopy. For this, HeLa cells were transfected with expression plasmids which contain the full-length sequence of the Bcl-rambo protein, that is amino acids 1-485, on the one hand, and, on the other hand, the Bcl-rambo protein sequence without the C-terminal transmembrane domain, that is only amino acids 1-459 (abbreviated to ΔMA). The location of the protein was determined 48 hours after the transfection, with the anti-Bcl-rambo antibody A1167 being used for this purpose. Prior to the fixing step, the transfected cells were incubated with Mitotracker. The photographs show (a) the superposition of Bcl-rambo fluorescence (green) and Mitotracker (red) in yellow, (b) Mitotracker on its own, (c) the location of anti-Bcl-rambo antibody on its own and (d) finally, a phase-contrast photograph. This experiment shows that the Bcl-rambo protein is essentially located in the cytosol and, in the cytosol, is located in intracellular organelles, as is also typical for other proteins in the Bcl-2 family. Comparable investigations carried out using transfected 293T cells gave identical results which are not shown in FIG. 3. The present colocation investigations make it clear that the Bcl-rambo protein essentially colocates with Mitotracker in the mitochondria. The investigations carried out using ΔMA, that is the Bcl-rambo protein without the C-terminal membrane anchor region, show that, in this case, the protein label has disappeared from the cytoplasm and it is possible to observe a diffuse nuclear localization for this polypeptide. It can be concluded from this that the location of the Bcl-rambo protein, which is evidently predominantly mitochondrial, is determined by its hydrophobic C-terminal anchor, as has already been reported for other proteins of the Bcl-2 family in regard to its importance for subcellular distribution.

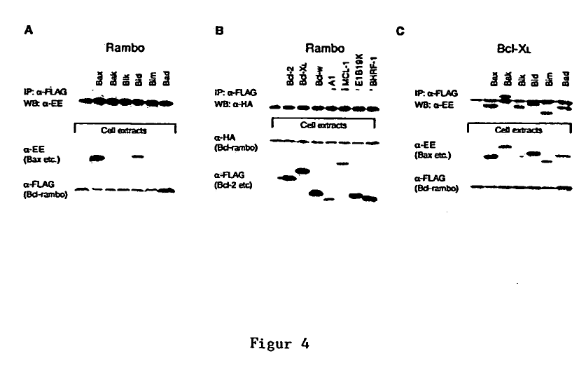

FIG. 4 shows the results of experiments relating to the question of the interaction of Bcl-rambo protein with various antiapoptotic and proapoptotic proteins of the Bcl-2 family, with appropriately transfected and Bcl-rambo-overexpressing 293T cells once again being used for this purpose. For this, 293T cells were transiently transfected with Flag-labeled Bcl-rambo protein and also either with a Flag-labeled, antiapoptotic protein of the Bcl-2 family (FIG. 4A) or an EE-labeled proapoptotic Bcl-2 in the presence of the caspase inhibitor z-VAD-fmk (FIG. 4B). Postnuclear lysates of these cells were immunoprecipitated using anti-Flag M2 agarose and blotted with anti-EE antibody. In this experiment, it was not possible to detect any protein/protein interaction between Bcl-rambo and the proapoptotic proteins Bax, Bak, Bik, Bid, Bim, Bad and Bcl-rambo (FIG. 4A). Results of corresponding experiments are shown in FIG. 4B, with HA-labeled Bcl-rambo being transfected, in these experiments, into 293T cells together with an antiapoptotic Flag-labeled protein of the Bcl-2 family. In this case, too, it can be seen that it was not possible to detect any interactions between Bcl-rambo and the antiapoptotic proteins (Bcl-2, Bcl-X_{L}, Bcl-w, A1, MCL-1, E1B-19K and BHRF1).

In order to demonstrate that the absence of interactions between Bcl-rambo and proapoptotic proteins of the Bcl-2 family was not due to the transfected cells dying at an elevated rate, the experiments were repeated in the presence of the caspase inhibitor z-VAD-fmk. As a pancaspase inhibitor, this inhibitor suppresses apoptotic events. The result found in FIG. 4A was reproduced in this case as well, which means that the lack of interaction between Bcl-rambo and the proteins investigated was not due to a systematic error. As shown in FIG. 4B, the possible interaction between Bcl-rambo and various antiapoptotic proteins of the Bcl-2 family, namely Bcl-2, Bcl-x_{L}, Bcl-w, A1, Mcl-1, EIB19K and Bhrf1, was investigated and no interactions were observed in this case, either. Without it being depicted in FIG. 4B, it was also possible to demonstrate that Bcl-rambo does not interact with other proteins of the Bcl-2 family even when the Bcl-rambo is only expressed in a truncated version, namely containing amino acids 1-223, that is only containing the region of homology with Bcl-2. Nor can any homodimers be detected in the case of Bcl-rambo.

FIG. 4C shows the result of a control experiment in which 293T cells were transfected, for 22 hours, with Flag-labeled Bcl-X_{L} and EE-labeled Bax, Bak, Bik, Bid, Bim or Bad in the presence of zVAD-fmk. The postnuclear lysates were immunoprecipitated with anti-Flag M2 agarose and blotted with anti-EE antibodies. In contrast to the Western blots depicted in FIGS. 4A and 4B, the Western blot (upper part of FIG. 4C) shows bands which reflect protein/protein interaction between Bcl-X_{L} and the abovementioned EE-labeled proteins. This means that there is an interaction between the abovementioned BH3-only proteins and Bcl-X_{L}.

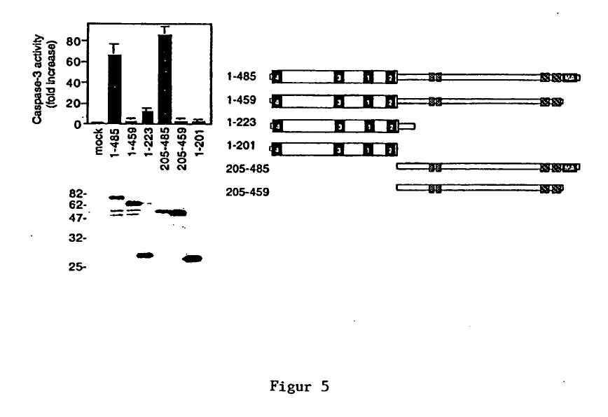

FIG. 5 describes the Bcl-rambo-induced apoptosis which is induced by the C-terminal domain of Bcl-rambo. For this, 293T cells were transfected with 2 μg of various Bcl-rambo constructs, namely mock transfectants, full-length Bcl-rambo (amino acids 1-485), Bcl-rambo without the C-terminal anchor domain (amino acids 1-459), the N-terminal domain of homology with Bcl-2 (amino acids 1-223), the C-terminal domain containing the BHNo region (amino acids 205-485), the C-terminal domain without the anchor region (amino acids 205-459) and, finally, a truncated N-terminal domain (amino acids 1-201). All the abovementioned constructs are depicted diagrammatically in FIG. 5 (right-hand drawing), with the BH regions (BH4 (4), BH3 (3), BH1 (1) and BH2 (2)) and also, if present, the tandem repeats and/or the anchor regions (MA) being shown diagrammatically. The transfection, which took place jointly together with 0.2 μg of an EGFP expression vector, namely pEGFP-C1, lasted for 42 hours. The graph shown in the upper left of FIG. 5 plots the caspase-3 activity (as the times increase in activity induced). The activity in the individual experimental assays was standardized by taking into account the fluorescence intensity of transfected EGFP, i.e. the cotransfection with the EGFP expression vector was used to check the transfection rate in order to ensure that it was possible to compare the results in the different experimental assays. The experiment showed that full-length Bcl-rambo had a marked proapoptotic effect (an approx. 70-fold increase in caspase-3 activity with subsequent cell death) with it being evident from the comparison with the effect of the mock transfectant that the proapoptotic effect of Bcl-rambo is specific. Thus, the mock transfectant does not have any adverse effect on the survival of the cell.

By contrast, overexpression of Bcl-rambo protein which encodes the N-terminal region of homology with Bcl-2, namely the constructs containing amino acids 1-223 or 1-203, has no effect on cell survival rate. By contrast, expression of the C-terminal BHNo domain (amino acids 205-459) resulted in an even greater increase in caspase-3 activation (an approx. 80-fold increase) as compared with the full-length Bcl-rambo protein even though the expression rates in the two experimental assays were identical. It follows from the results of the experiments depicted in FIG. 5 that the BHNo domain (and not, for example, the domain having homology with Bcl-2) is responsible for inducing cell death when Bcl-rambo is overexpressed. At the same time, the C-terminal transmembrane region must also be present since the 205-459 construct (that is the C-terminal domain without the anchor region) was no longer able to elicit cell death by activating caspase-3. In the case of all the cell extracts, the strength of the expression of the individual constructs in the transfected cells was checked by Western blot analysis using anti-Flag antibody M2 (lower left-hand diagram in FIG. 5).

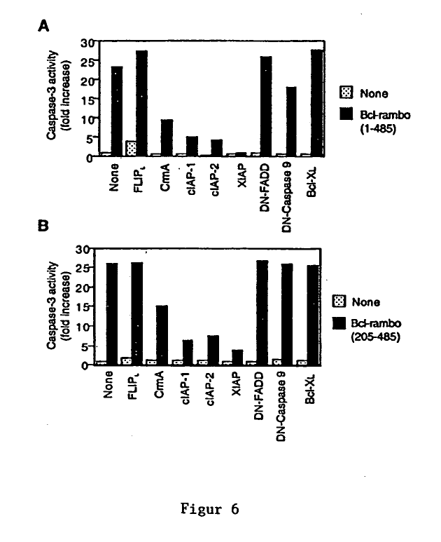

The effect of different apoptosis inhibitors on the apoptotic activity of Bcl-rambo is investigated in FIG. 6. For this, 293T cells were transfected for 27 hours (shown in FIG. 6A) with 2 μg of Bcl-rambo expression plasmid and 0.5 μg of expression vectors encoding various apoptosis inhibitors, together with 0.5 μg of β-galactosidase expression vector. The following apoptosis inhibitor proteins were selected: FLIP, CrmA, cIAP-1, cIAP-2, XIAP, DN-FADD, DN-caspase 9 and Bcl-X_{L}, and also a comparative experiment in which no vector expressing an apoptosis inhibitor was added (“none”). The increase in caspase-3 activity is plotted (as the times increase in activity, and standardized in accordance with the β-galactosidase activity which was also measured, for which reason it is possible to compare the individual experimental assays. The results given in FIG. 6 show that coexpression of cFLIP, a dominant-negative form of FADD, CrmA, Bcl-C1 and a dominant-negative form of caspase-9 is either unable to block Bcl-rambo-induced cell death or only able to block it marginally. Nevertheless, not depicted in FIG. 6, Bcl-rambo-induced cell death is caspase-dependent since it can be completely suppressed by the pancaspase inhibitor z-VAD-fmk. These results lead to the assumption that Bcl-rambo-induced cell death proceeds independently of a death receptor or the apoptotic cytochrome C-Apaf1-caspase-9 signal pathway. It can also be seen from FIG. 6A that an inhibitory effect is obtained after coexpression of xIAPs, and that the apoptotic inhibitors c-IAP1 and c-IAP2 also exhibit an inhibitory effect. It has already been previously demonstrated that the apoptosis inhibitors of the IAP family bind to caspases 3 and/or 6 and, to a lesser extent, to caspase 9, but not caspases 1, 6, 8 or 10, and inactivate these caspases.

According to the results shown in FIG. 6B, the apoptosis inhibitors behave toward the expression of transfected C-terminal Bcl-rambo, which encodes the BHNo domain and the membrane anchor domain (amino acids 205-485), in precisely the same way as they do toward the full-length protein (see FIG. 6A).

FIG. 7 shows the amino acid sequence of the human Bcl-rambo C-terminal domain, which also contains the BHNo domain. This is the sequence segment between amino acid 205 and amino acid 485 of the full-length human Bcl-rambo protein.

FIG. 8 shows the full-length amino acid sequence of Bcl-rambo, a protein comprising 485 amino acids. The cDNA sequence of the long form of human Bcl-rambo, containing all the exons (I-VI), is shown in FIG. 9.

The present invention is explained in more detail by means of the following implementation examples:

1^{st} Implementation Example

Identifying the Sequence of Bcl-Rambo

Carrying out a database search and using a specific search pattern to profile the investigation, led to the identification of the EST clone T48205, which, as it was subsequently to turn out, encodes a short splice variant of human Bcl-rambo. Following transfection, this splice variant exhibits the N-terminal amino acids 1-200 of the subsequently identified protein Bcl-rambo, which comprises a total of 485 amino acids. Another EST clone (AA190545) was identified, with this clone corresponding to the C-terminal region, containing amino acids 255 to 485, of the full-length Bcl-rambo protein. After that, a cDNA library was used to amplify other segments of the Bcl-rambo protein which were missing. As it was subsequently to turn out, these segments constituted the region containing amino acids 201-254 of Bcl-rambo. The missing region was amplified using the 5′ forward primer JT1108 (5′-CCT TCA CCA GCA CAG GCT TTG ACC G-3′) and the 3′ reverse primer JT1109 (5′GGA CCA CCT CCT CCA CTT CAG TAG G-3′). The PCR product resulting from this amplification was cloned in an anticlockwise direction in the PCR-blunt vector (from Invitrogen). The fragment which was specified by the cleavage sites for Not1 and BamH1 was then inserted into the corresponding Not1 and BamH1 restriction cleavage sites in the EST clone AA190545, thereby yielding a Bcl-rambo fragment which, as it subsequently turned out, corresponded to amino acids 78-485. Double PCR methods were carried out in order to arrive at the full-length Bcl-rambo protein. To do this, 2 DNA fragments were amplified, on the one hand from the plasmid carrying the Bcl-rambo fragment (amino acids 78-485) using the 5′ primer JT1145 (5′-CAT ACC TCG AGG ACT ATT C-3′) and the 3′ primer (flanked by the Not1 cleavage site) JT1144 (5′-TAG CGG CCG CCT ATT TCT TTC TCA G-3′) and, on the other hand, from the EST clone T48205 using the 5′ primer (flanked by the EcoRI cleavage site) JT1143 (5′-AAA GAA TTC ATG GCG TCC TCT TCT AC-3′) and the 3′ primer JT1146 (5′-GAA TAG TCC TCG AGG TAT G-3′). The two DNA fragments which resulted from these amplifications were mixed and amplified by PCR methods without primers, followed by an amplification using the primers JT1143 and JT1144. The PCR product was cloned into the PCR-blunt vector and the EcoRI/NotI fragment was then subcloned into a modified version of the PCR3 vector (from Invitrogen) in the same reading frame as an N-terminal Flag sequence or an HA peptide.

2^{nd} Implementation Example

Functional Analysis of the Bcl-Rambo Protein

(a) Constructing plasmids for functionally analyzing the full-length Bcl-rambo protein (amino acids 1-485):

Various constructs, which correspond to constituent sequences of the full-length protein, were prepared for this purpose.

1. Bcl-rambo comprising amino acids 1-201 (Bcl-rambo (1-201)): this construct was amplified by PCR methods from the EST clone T48205 using a 5′ forward primer, i.e. JT993, containing the EcoRI cleavage site (5′-AAA GAA TTC ATG GCG TCC TCT-3′) and the 3′ reverse primer JT994, containing a NotI cleavage site (5′-TAG CGG CCG CTC ATA CCC AGC CAC C-3′) and then cloned into the PCR-blunt vector.

2. The other deletion mutants of the full-length Bcl-rambo protein were constructed by means of PCR amplification employing the full-length Bcl-rambo protein as the template and using a 5′ forward primer, flanked by an EcoRI cleavage site, and a 3′ reverse primer, flanked by a NotI cleavage site, as follows:

2.1 Bcl-rambo comprising amino acids 1-223 (Bcl-rambo (1-223)): 5′ primer JT1143 and 3′ primer JT1244 (5′-AT AGC GGC CGC CTA GTC ATT GCT ATC TTC GT-3′),

2.2 Bcl-rambo comprising amino acids 1-459 (Bcl-rambo (1-459)): 5′ primer JT1143 and 3′ primer JT1245 (5′-AT AGC GGC CGC CTA AGA CTT GCC CTC AGA C-3′);

2.3 Bcl-rambo comprising amino acids 205-485 (Bcl-rambo (205-485)): 5′ primer JT1251 (5′-AAA GAA TTC AGT CTT GAG TCA GAG G-3′) and 3′ primer JT1144;

2.4 Bcl-rambo comprising amino acids 205-459 (Bcl-rambo 205-459)): 5′ primer JT1251 and 3′ primer JT1245.

The EcoRI/NotI fragments were subcloned into a modified version of the PCR3 vector containing an N-terminal Flag epitope.

(b) Cell Lines

The abovementioned constructs were used to transform cell lines, which were grown and cultured as described in the publication by Thome et al. ((1999) J. Biol. chem. 274(15), 9962-8). The entire content of the experiment description in the abovementioned publication is therefore part of the present disclosure.

(c) Investigations Performed on the Transfected Cells

1. Western Blotting Investigations

For this, approximately 10⁶ 293T cells per 10 cm culture dish were first of all transfected with the respective expression plasmids using the calcium phosphate/HEPES method, as described in Ausubel et al. ((1999) Introduction of DNA into Mammalian Cells. Current Protocols in Molecular Biology (Chanda, V. B. Ed.) 2.4 vols. John Wiley & Sons, Inc.). The entire content of the description of the method in Ausubel et al. ((1999) Introduction of DNA into Mammalian Cells. Current Protocols in Molecular Biology (Chanda, V.B.E.), 2.4 vols. John Wiley & Sons, Inc.) is therefore part of the present disclosure. 30 hours after the transfection, the cells were harvested and lyzed on ice in a complete NP40 lysis buffer. The NP40 lysis buffer corresponds to 1% Nonidet P-40 lysis buffer containing 20 mM Tris-HCl, pH 7.4, 150 mM NaCl and protease inhibitor cocktail (Complete, from Boehringer Mannheim).

For the subsequent immunoprecipitation, the postnuclear lyzates were first of all standardized with regard to their protein content (in order to be able to compare the individual measurement series), prepurified, at 4° C. for 2 hours, on Sepharose 6B (from Sigma) and only then subjected to immunoprecipitation, at 4° C. for at least 2 hours and on a rotating disk, using anti-Flag M2 agarose beads (from Sigma). The immunoprecipitates were washed twice with lysis buffer containing 1% and 0.05% NP-40, respectively and the washed beads were boiled in reducing sample buffer prior to loading onto SDS-PAGE and subsequent Western blotting.

For the Western blotting analysis, the immunoprecipitated proteins, which had in each case been labeled with either Flag, HA or EE labels, were detected using anti-Flag M2 antibody (from Sigma) (directed against Flag labels), using anti-VSV P5D4 antibody (from Sigma) (directed against the HA label) or using anti-EE antibody (from Eurogentec) (directed against EE-labeled proteins). Affinity-purified polyclonal rabbit antibodies (AL167) were used in cell lyzates (50 μg) in order to determine the cellular (that is endogenous) content of Bcl-rambo protein. AL167 antibody was prepared in rabbits against recombinant Bcl-rambo protein, with this antibody being specifically directed against the N-terminal moiety of the protein, comprising amino acids 1-223. Finally, the polyclonal antibody was then affinity-purified on immobilized antigen, as is familiar to the skilled person from the prior art.

Amino acids 1-223 tally with the corresponding segment of the human Bcl-rambo protein. The Western blots were then developed using peroxidase-coupled goat anti-mouse secondary antibody or peroxidase-coupled goat anti-rabbit secondary antibody (from Jackson ImmunoResearch Laboratories).

2. The Northern blots (from Clontech) were obtained by hybridization after approximately 3 hours, after using randomly labeled radioactive probes in ExpressHyb buffer (from Clontech, Palo Alto, Calif.); the blots were then washed, at room temperature for 40 minutes, with several changes of 2×SSC/0.1% SDS, followed by 0.1×SSC/0.05% SDS at 50° C. for 40 minutes.

(d) Investigations Relating to Apoptosis

293T cells (3×10⁵ cells) were sown on 60 mm culture dishes on the day before the transfection and then transiently cotransfected with 2 μg of Bcl-rambo expression plasmids and 0.2 μg of EGFP expression vector (pEGFP-C1, from Clontech) or with 0.5 μg of pCMV-β-galactosidase expression vector. The postnuclear lyzates were then analyzed with regard to the fluorescence intensity of EGFP, the galactosidase activity and the caspase-3 activity using a peptide substrate (Ac-DEVD-AMC, from Alexis). In order to be able to compare the individual measurements, the caspase-3 activity (measured as the number of times it was increased by the induction) was standardized using the fluorescence intensity of EGFP or the β-galactosidase activity.

(e) Immunolabeling and Confocal Laser Scanning Microscopy

On the day before the transfection, HeLa cells were plated out, at a confluence of 10-20%, on 20 ml glass cover slips in 5.5 cm culture dishes. On the following day, the cells were transfected using the calcium phosphate/BES method and then harvested approximately 36 hours after beginning the transfection. In order to ensure mitochondrial labeling, the cells were incubated for 30 minutes in a culture medium in the presence of 1 μM Mitotracker (from molecular probes). The cells on glass cover slips were then washed twice with cold PBS, fixed for 12 minutes in 4% paraformaldehyde at room temperature, washed twice with cold PBS and permeabilized overnight at 4° C. in PBS containing 0.1% saponin (PBSS). The glass cover slips were then blocked at room temperature for 30 minutes with 5% milk in PBSS (PBSSM), labeled with primary antibody AL167 (1 μg/ml) (anti-Bcl-rambo antibody) at room temperature for one hour, washed 3× with PBSS and labeled with Alexa 488-labeled (from molecular probes) secondary anti-mouse antibody or anti-rabbit antibody diluted 1/100 in PBSSM for one hour in the dark. Finally, the glass cover slips were washed three times with PBS and fixed on microscope slides using FluorSave reagent (from Calbiochem). The confocal microscopy was carried out on a Zeiss Axiovert 100 microscope (Zeiss Laser Scanning Microscope 510). In order to be able to detect Cy5 fluorochrome, a helium laser was filtered at 633 nm. On the other hand, an argon laser was used at 488 nm in order to be able to detect the Alexa fluorochrome. Standard conditions were chosen with regard to the aperture size for the imaging, with each image being a mean of 16 runs.

1. A DNA sequence, characterized in that it contains a sequence region which encodes a polypeptide having an amino acid sequence as depicted in FIG. 7, including all the functionally homologous derivatives, fragments or alleles, or DNA sequences which hybridize therewith.

2. A DNA sequence as claimed in claim 1, characterized in that it encodes a polypeptide as depicted in FIG. 7, including all the functionally homologous derivatives, fragments or alleles, or DNA sequences which hybridize therewith.

3. A DNA sequence as claimed in claim 1 or 2, characterized in that it contains a sequence region which encodes a polypeptide as depicted in FIG. 8, including all the functionally homologous derivatives, alleles or fragments, or DNA sequences which hybridize therewith.

4. A DNA sequence as claimed in one of the preceding claims, characterized in that it contains the (c)DNA sequence specified in FIG. 9.

5. An expression vector, characterized in that it contains a DNA sequence as claimed in one of claims 1 to 4.

6. A host cell, characterized in that it is transformed with an expression vector as claimed in claim 5.

7. A host cell as claimed in claim 6, characterized in that it is a mammalian cell, in particular a human cell.

8. A purified gene product, characterized in that it is encoded by a DNA sequence as claimed in one of claims 1 to 4.

9. A purified gene product as claimed in claim 8, characterized in that it is a polypeptide.

10. A purified gene product as claimed in claim 8 or 9, characterized in that it contains the amino acid sequence specified in FIG. 7, including all the functionally homologous alleles, fragments or derivatives.

11. An antibody, characterized in that it recognizes an epitope on a gene product as claimed in one of claims 8 to 10.

12. An antibody as claimed in claim 11, characterized in that it is monoclonal.

13. An antibody as claimed in claim 11 or 12, characterized in that it is directed against a sequence segment, as the epitope, on the BHNo domain.

14. A method for isolating gene products which at least contain an amino acid sequence as depicted in FIG. 7, including all the functionally homologous fragments, derivatives or alleles of this sequence, characterized in that host cells as claimed in claim 6 or 7 are cultured under suitable, expression-promoting conditions and the gene product is then purified from the culture.

15. A method for expressing gene products which at least contain an amino acid sequence as depicted in FIG. 7, including all the functionally homologous fragments, derivatives or alleles of this sequence, characterized in that host cells are transformed with an expression vector as claimed in claim 5.

16. The use of a DNA sequence as claimed in one of claims 1 to 4, or of a gene product as claimed in one of claims 8 to 10, for treating (or for producing a pharmaceutical for treating) diseases which are due to incorrectly regulated intracellular signal transduction.

17. The use of a DNA sequence or of a gene product as claimed in claim 16 for treating (or for producing a pharmaceutical for treating) diseases which are due to incorrectly regulated cell apoptosis, in particular a deficiency in apoptosis.

18. The use of a DNA sequence or of a gene product as claimed in claim 16 or 17 for treating (or for producing a pharmaceutical for treating) tumor diseases.

19. A compound, characterized in that it modulates, in particular inhibits, the intracellular function of Bcl-rambo.

20. A compound as claimed in claim 19, characterized in that it inhibits the Bcl-rambo-mediated activation of caspase-3.

21. A compound as claimed in claim 19 or 20, characterized in that it is an organochemical compound having a molecular weight of preferably <3000.

22. A compound as claimed in one of claims 19 to 21, characterized in that it passes through the cell membrane by diffusion or by way of membranous transport proteins.

23. A method for identifying pharmaceutically active compounds as claimed in one of claims 19 to 22, characterized in that
(a) a suitable host cell system is transfected with an expression vector as claimed in claim 5, in particular an expression vector which encodes the Bcl-rambo protein, at least one expression vector which encodes at least one apoptosis inhibitor, and, where appropriate, at least one expression vector which encodes at least one reporter gene, and
(b) measuring, for the host cell system obtained in accordance with (a), a parameter which is suitable for observing the Bcl-rambo-mediated apoptosis, in particular the activation of caspase-3, which occurs after adding a test compound as compared with the control without any addition of a test compound.

24. The method as claimed in claim 23, characterized in that the apoptosis inhibitor is FLIP, CrmA, DN-FADD and/or DN-caspase-9 and the reporter gene is β-galactosidase.

25. The use of a compound as claimed in claims 19 to 22 for treating (or for producing a pharmaceutical for treating) neurodegenerative diseases, in particular Alzheimer's dementia and Parkinson's disease, muscular dystrophy, viral infectious diseases and autoimmune diseases.

26. A method for identifying cellular interaction partners of the protein Bcl-rambo, with what is termed a yeast two-hybrid system being used.

27. The use of a DNA sequence as claimed in one of claims 1 to 4, or of a gene product as claimed in one of claims 8 to 10, for identifying other proteins involved in apoptotic signal transduction.

28. The use of a DNA sequence as claimed in one of claims 1 to 4, or of a gene product as claimed in one of claims 8 to 10, as a suicide gene/suicide protein for transforming host cells in vivo or ex vivo.

29. The use of a DNA sequence or of a gene product as claimed in claim 28, with the suicide gene being operably linked to a promoter, and with the transcription being repressed and only activated when needed.
